(19) United States
(12) Patent Application Publication
(10) Pub. No.: US 2010/0004257 A1
(43) Pub. Date: Jan. 7, 2010

(54) Identification of Biomarkers Predictive of Dasatinib Effects in Cancer Cells

(75) Inventor: Eric Bruce Haura, Tampa, FL (US)

Correspondence Address: SMITH HOPEN, PA, 180 PINE AVENUE NORTH, OLDSMAR, FL, 34677, US

(73) Assignee: University of South Floria, Tampa, FL (US)

(21) Appl. No.: 12/501,897

(22) Filed: Jul. 13, 2009

Related U.S. Application Data
(63) Continuation of application No. PCT/US2008/050994, filed on Jan. 14, 2008.
(60) Provisional application No. 60/884,634, filed on Jan. 12, 2007.

(51) Int. Cl. A61K 31/506; A61P 35/00; A61P 35/02

(52) U.S. Cl. 514/252.19

(57) ABSTRACT

A method of predicting response to treatment with inhibitors of EGFR and SRC by screening for status of key biomarkers such as EGFR. Dasatinib is a drug that can inhibit a group of proteins called SRC proteins. In addition, other experiments have suggested that other important signaling proteins are affected by dasatinib. Early phase trials of dasatinib are ongoing in cancer patients. It will be important to determine which patients receive a clinical benefit of dasatinib. Predetermination of treatment benefit can be performed by assessing biomarkers in patients tumors prior to treatment with dasatinib or other inhibitors of EGFR and SRC. Patients that have positive biomarkers for treatment could then be treated with higher confidence of benefit while those not possessing these predictive biomarkers would avoid ineffective and potentially toxic therapy. Additionally, treatment can be tailored according to predetermined sensitivity by evaluating indicated biomarkers correlating with sensitivity to one or more agents.

### CROSS REFERENCE TO RELATED APPLICATIONS

This application is a continuation of prior filed International Application, Serial Number PCT/US2008/50994 filed Jan. 14, 2008, which claims priority to currently pending U.S. Provisional Patent Application 60/884,634, entitled, “Identification of Biomarkers Predictive of Dasatinib Effects in Lung Cancer Cells”, filed Jan. 12, 2007, the contents of which are herein incorporated by reference.

### STATEMENT OF GOVERNMENT INTEREST

This invention was made with Government support under Grant Nos. CA055652 and CA082533 awarded by the National Institutes of Health. The Government has certain rights in the invention.

### FIELD OF INVENTION

This invention relates to cancer therapy. More specifically, this invention relates to biomarkers predictive of dasatinib effects in cancer cells.

### BACKGROUND AND SUMMARY OF THE INVENTION

Activating mutations in the tyrosine kinase domain of the epidermal growth factor (EGF) receptor (EGFR) selectively activate Akt and signal transducer and activator of transcription (STAT) pathways important in lung cancer cell survival. Cell lines harboring activated EGFR molecules are dependent on EGFR for survival because inhibition of EGFR results in apoptosis.

Src family kinases can link signals originating from growth factor, integrin, and cytokine receptors on the surface of cells to their downstream effector signaling cascades. c-Src cooperates with EGFR and ErbB2 and can be necessary for transformation by EGFR. In addition, c-Src directly modulates EGFR function through phosphorylation of tyrosine residues on EGFR that allows for coupling to downstream signaling events. Src family kinases can cooperate with receptor tyrosine kinases to signal through downstream molecules, such as phosphatidylinositol 3-kinase (PI3K)/PTEN/Akt and STATs.

Dasatinib is a small molecule inhibitor of SRC and other tyrosine kinases implicated in the biology of cancer. SRC specifically can affect “hallmark” pathways of cancer including those that regulate cell growth, survival, invasion/metastasis, and angiogenesis. We have been investigating the effects of dasatinib on cell growth, survival, and invasion in a collection of non-small cell lung cancer cell lines.

Dasatinib is a drug that can inhibit a group of proteins called SRC proteins. In addition, other experiments have suggested that other important signaling proteins are affected by dasatinib. Early phase trials of dasatinib are ongoing in cancer patients. What is needed is a method to determine which of these patients will benefit from treatment with inhibitors of EGFR and SRC proteins. Patients that will potentially benefit from treatment could then be treated with higher confidence of benefit, while those not likely to benefit would avoid ineffective and potentially toxic therapy. It would be highly desirable to have a method predictive of sensitivity to treatment of dasatinib. It would also be desirable to have a method of predicting sensitivity to combination therapy with dasatinib and one or more EGFR inhibitors such as gefitinib and erlotinib. The present invention solves this and other important needs as will be evident in the specification below.

### SUMMARY OF THE INVENTION

A method of predicting response to treatment with inhibitors of EGFR and SRC by screening for status of key biomarkers such as EGFR. In accordance with the invention, the problem of predicting response to treatment with inhibitors of EGFR and SRC is solved by a method of screening cancer cells of the subject to determine the EGFR status of the cells. EGFR status refers to the status of the EGFR in the cell or cells under examination, or more particularly, the presence or absence of activating mutations or additions in EGFR of the cell and/or the identity of the particular mutation or addition in the EGFR. The EGFR status of the cell can then be used to predict the sensitivity of the cell to treatment with inhibitors of EGFR and SRC. The inventors have discovered that, using such methodology, it is possible to correlate the benefit of treatment with key compounds, such as the SRC inhibitor dasatinib, with the EGFR status of the cell.

Dasatinib is a drug that can inhibit a group of proteins called SRC proteins. In addition, other experiments have suggested that other important signaling proteins are affected by dasatinib. It will be important to determine which patients receive a clinical benefit of dastinib. Predetermination of treatment benefit can be performed by assessing biomarkers in patients tumors prior to treatment with dasatinib or other inhibitors of EGFR and SRC. Patients that have positive biomarkers for treatment could then be treated with higher confidence of benefit while those not possessing these predictive biomarkers would avoid ineffective and potentially toxic therapy. Additionally, treatment can be tailored according to predetermined sensitivity by evaluating indicated biomarkers correlating with sensitivity to one or more agents.

In a first aspect the present invention provides a method of treating a proliferative disorder in a subject. The method includes the steps of screening cells of the subject to determine the EGFR status of the cells, correlating the EGFR status of the subject's cells to the dasatinib treatment sensitivity associated with the EGFR status, and administering a therapeutically-effective amount of dasatinib, or a pharmaceutically-acceptable derivative thereof, to the subject responsive to the correlated EGFR status of the subject. In certain advantageous embodiments the sensitivity of cells possessing EGFR status biomarker is determined prior to the screening step. Based upon the results of the screening and correlating, the dosage of dasatinib to be administered to the cancer cell population responsive to the correlation of the EGFR status of the subject's cells can be adjusted. The dasatinib can be orally administered. The method of treating a proliferative disorder in a subject can further include the administration of one or more additional therapeutic agents. Additional therapeutic agents can include gefitinib, erlotinib and combinations thereof.

The proliferative disorder can be a disease such as lung cancer, including small-cell lung cancer, non-small cell lung cancer, ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, immunocompromised status resulting in increased susceptibility to lung infections, bacterial pneumonia, viral pneumonia, mycoplasma pneumonia, fungal pneumonia, Legionnaires' Disease, Chlamydia pneumonia, aspiration pneumonia, Nocordia sp. Infections, parasitic pneumonia, necrotizing pneumonia.

In an advantageous embodiment the proliferative disorder is leukemia, breast cancer, prostate cancer, lung cancer, colon cancer, melanoma, or solid tumors. The leukemia can include treatment for T-cell acute lymphoblastic leukemia (T-ALL), chronic myeloid leukemia (CML), Ph+ ALL, AML, imatinib-resistant CML, imatinib-intolerant CML, accelerated CML, and lymphoid blast phase CML.

In further advantageous embodiments the proliferative disorder may be a proliferative disorder of the lung. Proliferative disorders of the lung include lung cancer, small-cell lung cancer, non-small cell lung cancer, ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, immunocompromised status resulting in increased susceptibility to lung infections, bacterial pneumonia, viral pneumonia, mycoplasma pneumonia, fungal pneumonia, Legionnaires' Disease, Chlamydia pneumonia, aspiration pneumonia, Nocordia sp. infections, parasitic pneumonia, necrotizing pneumonia.

In a second aspect the present invention provides a method of treating lung cancer in a subject. The method includes the steps of screening cancer cells of the subject to determine the EGFR status of the cells, correlating the EGFR status of the subject's cells to the dasatinib treatment sensitivity associated with the EGFR status and administering a therapeutically-effective amount of dasatinib to the subject responsive to the correlated EGFR status of the subject's cells. In certain advantageous embodiments the sensitivity of cells possessing EGFR status biomarker is determined prior to the screening step. In other words, the sensitivity of cells to treatment with dasatinib associated with a defined EGFR status can be determined prior to the screening step using a control cell population. The control cell population will have a known, defined mutation, addition or other status. By predetermining the sensitivity associated with a particular biomarker, a rapid correlation of sensitivity the cancer cell population following the screening step can be achieved, allowing treatment to begin more readily. Based upon the results of the screening and correlating, the dosage of dasatinib to be administered to the cancer cell population responsive to the correlation of the EGFR status of the subject's cells can be adjusted. In this manner, treatment can be tailored to meet the particular needs of the subject. In a particularly advantageous embodiment, the lung cancer is non-small cell lung cancer.

The method of treating lung cancer in a subject can further include the administration of one or more additional therapeutic agents. Additional therapeutic agents can include gefitinib, erlotinib and combinations thereof.

The dasatinib can be administered orally. In certain embodiments the effective amount of dasatinib ranges between about 35 and about 150 mg. dasatinib per patient per day.

In a third aspect the present invention provides a method of treating a proliferative disorder, such as lung cancer, in a subject. The method includes the steps of screening cancer cells of the subject to determine the EGFR status of the cells, correlating the EGFR status of the subject's cells to the SRC tyrosine inhibitor treatment sensitivity associated with the EGFR status and administering a therapeutically-effective amount of an SRC tyrosine inhibitor the subject responsive to the correlated EGFR status of the subject's cells. The SRC tyrosine kinase inhibitor can be dasatinib.

In a fourth aspect the present invention provides a method of treating cancer in a subject. The method includes the steps of screening cancer cells of the subject to determine the EGFR status of the cells, correlating the EGFR status of the subject's cells to the dasatinib treatment sensitivity associated with the EGFR status and administering a therapeutically-effective amount of dasatinib to the subject responsive to the correlated EGFR status of the subject's cells. In certain advantageous embodiments the sensitivity of cells possessing EGFR status biomarker is determined prior to the screening step. The dosage of dasatinib administered can be adjusted according to the correlated sensitivity of the cell. In an advantageous embodiment the cancer is leukemia, breast cancer, prostate cancer, lung cancer, colon cancer, melanoma, or solid tumors. The leukemia can include treatment for T-cell acute lymphoblastic leukemia (T-ALL), chronic myeloid leukemia (CML), Ph+ ALL, AML, imatinib-resistant CML, imatinib-intolerant CML, accelerated CML, and lymphoid blast phase CML.

In an advantageous embodiment the method can include administering an additional therapeutic agent or treatment regimen in combination with dasatinib treatment. The one or more additional agents can be an EGFR inhibitor. In a particularly advantageous embodiment the EGFR inhibitor is gefitinib, erlotinib and combinations thereof.

In further advantageous embodiments the method of treating cancer in a subject can include the steps of correlating a biomarker of the screened cancer cells with the sensitivity of cells possessing that biomarker to treatment one or more EGFR inhibitors and administering the one or more EGFR inhibitors in combination with dasatinib. In certain embodiments both biomarkers are biomarkers of EGFR status. In certain embodiment the administration of the one or more EGFR inhibitors can include adjusting the dosage of the EGFR inhibitors according to the EGFR status or other biomarker status of the cell. In alternative embodiments the dosage of the one or more EGFR inhibitors can be administered according to the correlated dasatinib sensitivity of the call.

In a fifth aspect the present invention provides a method of assessing the sensitivity of a cancer cell population to treatment with dasatinib or other SRC inhibitor. The method includes screening the cancer cells for one or more biomarkers indicative of sensitivity to dasatinib other SRC inhibitor and correlating the biomarker of the screened cancer cells with the sensitivity of cells possessing that biomarker to treatment with dasatinib or other SRC inhibitor. The screened biomarker can be a biomarker indicative of EGFR status. The cancer cell population can be a lung cancer cell population.

In a sixth aspect the present invention provides a method of treating cancer in a subject including the step of comprising the step of administering a combination of erlotinib and dasatinib to a patient in need of treatment. The patient can be a patient exhibiting resistance to erlotinib treatment. In an advantageous embodiment the cancer is leukemia, breast cancer, prostate cancer, lung cancer, colon cancer, melanoma, or solid tumors. The leukemia can include treatment for T-cell acute lymphoblastic leukemia (T-ALL), chronic myeloid leukemia (CML), Ph+ ALL, AML, imatinib-resistant CML, imatinib-intolerant CML, accelerated CML, and lymphoid blast phase CML. In a particularly advantageous embodiment the cancer is non-small cell lung cancer. In further advantageous embodiments the subject has a defined EGFR status. The combination treatment can be administered according to the subject's EGFR status.

In a seventh aspect the present invention provides a method of treating lung cancer in a subject including the steps of screening cancer cells of the subject to determine sensitivity to one or more EGFR tyrosine kinase inhibitors and administering a therapeutically-effective amount of an SRC inhibitor to the subject responsive to the sensitivity to one or more EGFR tyrosine kinase inhibitors of the subject's cells. It is found that sensitivity to one or more EGFR tyrosine kinase inhibitors correlates with sensitivity to one or more SRC inhibitors. One of the one or more EGFR tyrosine kinase inhibitors can be erlotinib. One of the one or more SRC inhibitors can be dasatinib.

In an eighth aspect the present invention provides a method of assessing the sensitivity of a cancer cell population to treatment with erlotinib. The method includes the steps screening the cancer cells for one or more biomarkers of EGFR status and correlating the biomarker of the screened cancer cells with the sensitivity of cells possessing that biomarker to treatment with erlotinib.

In an ninth aspect the present invention provides a method for the treatment of cancer in a patient resistant to treatment with erlotinib. The method includes the step of administering dasatinib to the erlotinib-resistant patient in need of treatment.

### BRIEF DESCRIPTION OF THE DRAWINGS

For a fuller understanding of the invention, reference should be made to the following detailed description, taken in connection with the accompanying drawings, in which:

FIG. 1 illustrates the effect of dasatinib on cell viability in lung cancer cell lines with various EGFR mutations. The figure presents a graph illustrating cell viability assay for cell lines with both mutant EGFR(H3255, H1650, HCC827, PC9, and H1975) and WT EGFR(H460, H1299, A549, and H358). Cells were exposed to the indicated concentration of dasatinib, and cell viability was assayed after 72 hours. The group of line plots labeled a, represents cell lines H460, H1299, A549, H1975 and H358. The group of line plots labeled a₂ represents cell lines H3255, H1650, HCC827, and PC9.

FIG. 2 further illustrates the effect of dasatinib on cell viability in lung cancer cell lines with various EGFR mutations. A parallel group of H1650 cells was treated with the indicated concentrations of dasatinib (BMS), and whole-cell lysates were subjected to Western analysis with pSrc and c-Src antibodies.

FIG. 3 further illustrates the effect of dasatinib on cell viability in lung cancer cell lines with various EGFR mutations. The figure shows a Western blot analysis of activated EGFR, Src, STAT3, and Akt in mutant and WT EGFR cell lines. Whole-cell lysates were prepared from untreated cells grown in 5% BCS and subjected to Western blot analysis with indicated antibodies. Antibodies included pEGFR and total EGFR, pSTAT3 and total STAT3, and pAkt and total Akt. Global activity of Src family kinases was also evaluated with pSrc antibody that recognizes phosphorylated forms of all nine Src members.

FIG. 4 further illustrates the effect of dasatinib on cell viability in lung cancer cell lines with various EGFR mutations. The figure presents a graph illustrating where cells were treated with 500 nmol/L of dasatinib and effects on apoptosis (Apo-BrdU incorporation) and assayed after 36 hours.

FIG. 5 further illustrates the effect of dasatinib on cell viability in lung cancer cell lines with various EGFR mutations. The figure presents a graph illustrating where indicated cells were treated with 500 nmol/L dasatinib for 16 to 24 hours (before onset of gross DNA fragmentation), and effect on cell cycle was assayed using propidium iodide staining and flow cytometry.

FIG. 6 illustrates that dasatinib induces apoptosis in EGFR-mutant NSCLC through down-regulation of Akt and STAT3. FIG. 6. presents a series of four graphs, with each graph representative of a different cell line as indicated at the top of the graph. The indicated cells were exposed to increasing concentrations of gefitinib or dasatinib, and cell viability was assessed at 72 hours.

FIG. 7 further illustrates that dasatinib induces apoptosis in EGFR-mutant NSCLC through down-regulation of Akt and STAT3. Mutant EGFR cell lines were exposed to indicated concentrations of either gefitinib or dasatinib, and total proteins were collected after 24 hours. Membranes were blotted with indicated antibodies.

FIG. 8 further illustrates that dasatinib induces apoptosis in EGFR-mutant NSCLC through down-regulation of Akt and STAT3. The indicated cells were exposed to increasing concentrations of either dasatinib or gefitinib for 24 hours, and whole-cell lysates were evaluated for pSrc and total c-Src using Western analysis. H1650 cells were grown in either 5% BCS or 0.5% BCS plus supplemental 100 ng/mL EGF. ZD, gefitinib.

FIG. 9 illustrates the effect of dasatinib and gefitinib on EGFR phosphorylation. Cells were exposed to indicated concentrations of dasatinib, and total proteins were collected after 24 hours. Membranes were blotted with indicated antibodies.

FIG. 10 further illustrates the effect of dasatinib and gefitinib on EGFR phosphorylation. HEK293 cells were transfected with plasmids encoding indicated EGFR cDNA and exposed to either dasatinib (500 nmol/L) or gefitinib (1 μmol/L) for 3 hours. Whole-cell lysates were prepared and subjected to Western analysis with indicated antibodies. C, control (DMSO).

FIG. 11 illustrates the comparative effect of dasatinib and gefitinib on cell cycle progression and tumor cell invasion. A549 and H358 cells were exposed to 1 μmol/L gefitinib, 500 nmol/L dasatinib, or the combination (ZD+BMS) for 3 hours, and whole-cell lysates were subjected to Western analysis using indicated antibodies.

FIG. 12 presents a pair of graphs further illustrating the comparative effect of dasatinib and gefitinib on cell cycle progression and tumor cell invasion. A549 and H358 cells were exposed to 1 umol/L gefitinib, 500 nmol/L dasatinib, or the combination (ZD⁺ BMS) for 24 hours, and cell cycle profiles were evaluated.

FIG. 13 further illustrates the comparative effect of dasatinib and gefitinib on cell cycle progression and tumor cell invasion. A549 and H358 cells were exposed to 1 pmoVL gefitinib, 500 nmol/L dasatinib, or the combination (ZD+BMS) for 24 hours, and cell cycle profiles were evaluated. Parallel group of cells was evaluated for changes in cyclins D1 and D3 and p27 by Western analysis.

FIG. 14 presents a graph further illustrating the comparative effect of dasatinib and gefitinib on cell cycle progression and tumor cell invasion. The indicated cells were exposed to 1 μmol/L gefitinib or 500 nmol/L dasatinib, and tumor cell invasion was quantified using Boyden chambers.

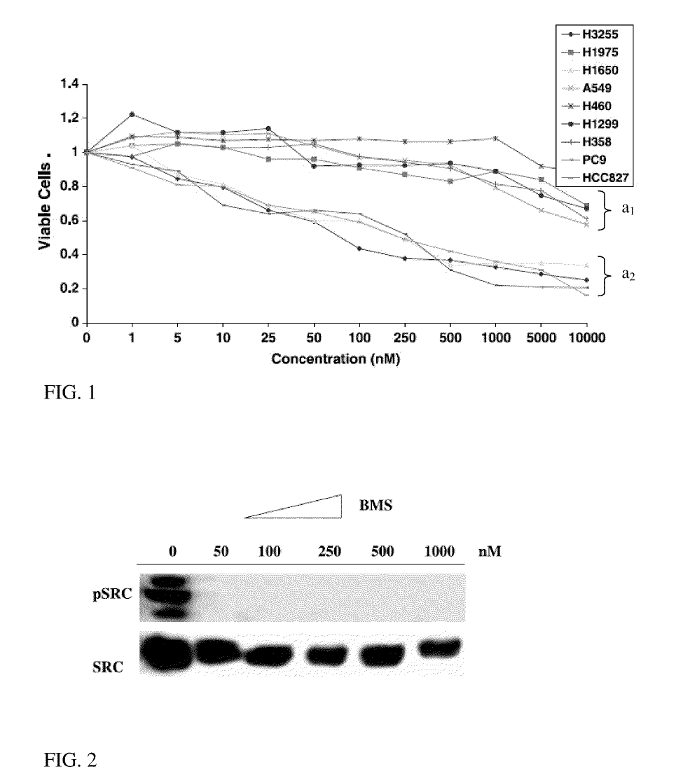

### DETAILED DESCRIPTION OF THE PREFERRED EMBODIMENT

Mutations of the epidermal growth factor receptor (EGFR) selectively activate Akt and signal transducer and activator of transcription (STAT) pathways that are important in lung cancer cell survival. Src family kinases can cooperate with receptor tyrosine kinases to signal through downstream molecules, such as phosphatidylinositol 3-kinase/PTEN/Akt and STATs. Based on the importance of EGFR signaling in lung cancer, the known cooperation between EGFR and Src proteins, and evidence of elevated Src activity in human lung cancers, we evaluated the effectiveness of a novel orally bioavailable Src inhibitor dasatinib (BMS-324825) in lung cancer cell lines with defined EGFR status. Here, it is shown that cell fate (death versus growth arrest) in lung cancer cells exposed to dasatinib is dependent on EGFR status. In cells with EGFR mutation that are dependent on EGFR for survival, dasatinib reduces cell viability through the induction of apoptosis while having minimal apoptotic effect on cell lines with wild-type (WT) EGFR. The induction of apoptosis in these EGFR-mutant cell lines corresponds to down-regulation of activated Akt and STAT3 survival proteins. In cell lines with WT or resistant EGFR mutation that are not sensitive to EGFR inhibition, dasatinib induces a G1 cell cycle arrest with associated changes in cyclin D and p27 proteins, inhibits activated FAK, and prevents tumor cell invasion. The results show that dasatinib can be effective therapy for patients with lung cancers through disruption of cell growth, survival, and tumor invasion. The results indicate EGFR status is important in deciding cell fate in response to dasatinib.

The data indicate that the decision fork for apoptosis versus growth arrest in cells exposed to dasatinib is dependent on the degree of upstream EGFR dependence for survival. Dasatinib shuts down the EGFR-dependent survival network in a concentration-dependent manner and induces death in EGFR-dependent cells. Dasatinib-induced apoptosis has been observed in head and neck cancer cells, another EGFR-dependent tumor type (Johnson F M, et al. Clin Cancer Res 2005; 11:6924-32). Mechanisms of dasatinib-induced apoptosis in gefitinib-sensitive mutant EGFR lung cancer cells are under study. In addition to Src proteins, dasatinib has been shown to bind other tyrosine kinase proteins, including EGFR, and, in conjunction with the data presented herein, one explanation is that EGFR may be a direct target of dasatinib or an indirect target secondary to Src inhibition (Ishizawar R, et al., Cancer Cell 2004; 6:209-14; Bromann P A, et al. Oncogene 2004; 23:7957-68; Carter T A, et al., Proc Natl Acad Sci 2005; 102:11011-6). In addition, Src signaling can regulate the PI3K/PTEN/Akt axis through multiple mechanisms, including tyrosine phosphorylation of the regulatory p85 subunit of PI3K, tyrosine phosphorylation of PTEN that results in compromised function of PTEN, and modification of EGFR function through direct phosphorylation of key tyrosine residues (Ishizawar R, et al., Cancer Cell 2004; 6:209-14; Bromann P A, et al. Oncogene 2004; 23:7957-68; Martin G S, Nat Rev Mol Cell Biol 2001; 2:467-75). Evidence indicates that Src proteins can directly phosphorylate STAT3 (Yu H and Jove R. Nat Rev Cancer 2004; 4:97-1059). Inhibition of pSTAT3 with dasatinib was only observed in the present studies in H3255 cells. This may be a direct effect on Src inhibition or it may be through modification of EGFR function. Nonetheless, the results suggest that Src is not responsible for high levels of activated STAT3 seen in cells with EGFR mutation. Other Src tyrosine kinase inhibitors (TKI) may produce similar effects on lung cancer cells with activating mutations in EGFR.

The effect of dasatinib in additional cells with acquired resistance to EGFR-TKI is a further area of interest. Despite dramatic responses in the subset of lung cancer patients with EGFR dependence, tumor cells may acquire resistance to EGFR-TKI therapy through either additional mutations in EGFR or other mechanisms (Haber D A and Settleman J. Cell Cycle 2005; 4:1057-919). Multiple inhibitors have been applied to overcome acquired resistance to TKIs in BCR-ABL-dependent leukemia, and a similar strategy may be explored in the treatment of EGFR-dependent lung cancer (Sawyers C L. Nat Med 2005; 11:824-5). Thus, combined attack on EGFR-dependent survival pathways by multiple nonoverlapping agents may be necessary to cure this subset of patients by avoiding the development of resistant clones. One possibility is that dasatinib added to EGFR-TKI may help suppress development of resistant clones, but this obviously requires further testing. The results herein show no apoptotic effect of dasatinib on H1975 cells with the T790M mutation, but this mechanism of resistance may be rare. Further evaluation in other cell lines that have acquired resistance to EGFR-TKI is indicated.

Dasatinib may have advantages over EGFR inhibitors in tumors that are not dependent on EGFR for survival through promoting tumor cell dormancy through cell cycle arrest and inhibition of tumor cell invasion. This is important because the majority of patients with advanced lung cancer do not have EGFR mutation. Because Src signaling is implicated in oncogenic processes, such as cell invasion, metastasis, and angiogenesis, these compounds could have additional in vivo effects beyond the effects seen in these cell culture models. A priori determination of lung cancers dependent on EGFR for growth and/or survival will identify patient subsets that derive the maximum benefit from dasatinib, and combination therapy with EGFR inhibitors should be considered.

Early phase trials of dasatinib are ongoing in cancer patients. The determination of which patients receive a clinical benefit of dastinib can be performed by assessing biomarkers in patient's tumors prior to treatment with dasatinib. Patients that have positive biomarkers for dasatinib could then be treated with higher confidence of benefit while those not possessing these predictive biomarkers would avoid ineffective and potentially toxic therapy. We have defined a number of such biomarkers and continue to develop additional markers predictive of treatment efficacy using laboratory models of lung cancer cells. In addition to dasatinib, cells or cell lines harboring activating EGFR mutations may show increased sensitivity to other Src inhibitors based upon the effects observed herein with dasatinib. We evaluated the antitumor efficacy of a novel orally bioavailable Src inhibitor dasatinib (BMS-354825) in cell lines with defined EGFR status, including wild-type (WT) and mutant EGFR sensitive to gefitinib. In addition to Src proteins, dasatinib can potentially interact with other important tyrosine kinase proteins involved in tumor cell growth and survival and these interactions could enhance its antitumor activity). We have identified biomarkers that are predictive of the effects of dasatinib in these lung cancer cells. Specifically, it is reported herein that activating mutations in the epidermal growth factor receptor (EGFR) predict sensitivity of the cells to dasatinib. In addition, activation of SRC measured by a phosphorylated SRC antibody should also be predictive of the effects of dasatinib in these cells. In addition, proteomics analysis of these cells should provide additional biomarkers that predict sensitivity to dasatinib.

Dasatinib is the generic name for the compound N-(2-chloro-6-methylphenyl)-2-[[6-[4-[(2-hydroxyethyl)-1-pipperazinyl]-2-methyl-4-pyrimidinyl]amino]-5-thiazolecarboxamide, monohydrate, also known as BMS-354825 and SPRYCEL, of the following formula I:

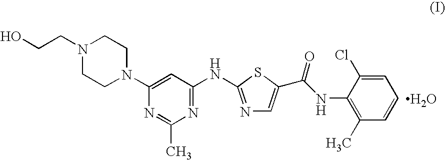

The compounds of Formula I may be prepared by the procedures described in PCT publication, WO 00/62778 published Oct. 26, 2000. The compound of formula I may be administered as described therein or as described in WO2004/085388, or as further described below with respect to the treatment of cancer/lung cancer.

Use of the term “N-(2-chloro-6-methylphenyl)-2-[[6-[4-(2-hydroxyethyl)-1-piperazinyl]-2-methyl-4-pyrimidinyl]amino]-5-thiazolecarboxamide” or its generic “dasatinib” encompasses (unless otherwise indicated) solvates (including hydrates) and polymorphic forms of the compound (I) or its salts (such as the monohydrate form of (I) described in U.S. Ser. No. 11/051,208, filed Feb. 4, 2005, incorporated herein by reference in its entirety and for all purposes).

Methods for treating an individual suffering from a proliferative disorder of the lung can comprise the steps of determining whether a biological sample obtained from the individual comprises wild type EGFR or an EGFR-dependent mutation, wherein the presence of the wild type EGFR is indicative of the individual being at least partially resistant to therapy, or at least having an increased likelihood of achieving a lower level of efficacy, with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, and administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, to the individual. The therapeutically effective amount will depend upon whether or not the individual has wild type EGFR and whether or not the therapy with N-(2-chloro-6-methylphenyl)-2-[[6-[4-(2-hydroxyethyl)-1-piperazinyl]-2-methyl-4-pyrimidinyl]amino]-5-thiazolecarboxamide will be combined with a second therapy. Currently, the recommended dosage for N-(2-chloro-6-methylphenyl)-2-[[6-[4-(2-hydroxyethyl)-1-piperazinyl]-2-methyl-4-pyrimidinyl]amino]-5-thiazolecarboxamide is twice daily as a 70 mg tablet, or 100 mg once daily, referred to as SPRYCEL®. In certain embodiments, if an individual is determined to have wild type EGFR that renders cells partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide treatment, the dosage of the drug can be increased. Alternatively, the drug can be administered in combination with a second therapy for treating the proliferative disorder of the lung. The second therapy can be any therapy effective in treating the disorder, including, for example, therapy with another protein kinase inhibitor such as imatinib, AMN107, PD180970, GGP76030, AP23464, SKI 606, NS-187, and/or AZD0530; therapy with a tubulin stabilizing agent for example, pacitaxol, epothilone, taxane, and the like; therapy with an ATP non-competitive inhibitor such as ONO12380; therapy with an Aurora kinase inhibitor such as VX-680; therapy with a p38 MAP kinase inhibitor such as BIRB-796; or therapy with a farnysyl transferase inhibitor. The dosage of N-(2-chloro-6-methylphenyl)-2-[[6-[4-(2-hydroxyethyl)-1-piperazinyl]-2-methyl-4-pyrimidinyl]amino]-5-thiazolecarboxamide treatment or a pharmaceutically acceptable salt, hydrate, or solvate thereof can remain the same, be reduced, or be increased when combined with a second therapy.

Individuals harboring EGFR activating mutations, or mutations that make the cells dependent upon EGFR, have an increased likelihood of achieving a desirable efficacious response, and thus administration of the typical prescribed dose of Dasatinib may be warranted.

The methods of treating a proliferative disorder of the lung in an individual will ideally inhibit proliferation of cancerous cells and/or induce apoptosis of the cancerous cells.

The individual to be screened or treated by the methods herein can be one that has received administration of a first kinase inhibitor to which the cancer cells in said individual have become resistant or at least partially resistant. The kinase inhibitor can be imatinib, N-(2-chloro-6-methylphenyl)-2-[[6-[4-(2-hydroxyethyl)-1-piperazinyl]-2-methyl-4-pyrimidinyl]amino]-5-thiazolecarboxamide, another kinase inhibitor, or any combination thereof. Alternatively, the individual will have not yet had treatment with a protein kinase inhibitor.

Combinations treatments comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are described in U.S. Ser. No. 10/886,955, filed Jul. 8, 2004, U.S. Ser. No. 11/265,843, filed Nov. 3, 2005, and U.S. Ser. No. 11/418,338, filed May 4, 2006, each of which are incorporated herein by reference in their entirety and for all purposes.

The invention comprises methods of establishing a treatment regimen for an individual having a proliferative disorder of the lung. The treatment regimen can comprise the administration of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, at a higher dose or dosing frequency than recommended for an individual having a mutated or activating EGFR. Alternatively, the treatment regiment can comprise combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide and any other agent that works to inhibit proliferation of cancerous cells or induce apoptosis of cancerous cells, including, for example, a tubulin stabilizing agent, a farnysyl transferase inhibitor, a BCR-ABL T315I inhibitor and/or another protein tyrosine kinase inhibitor. Preferred other agents include imatinib, AMN107, PD180970, CGP76030, AP23464, SKI 606, NS-187, or AZD0530. Also included are ATP non-competitive inhibitors, including, for example, ON012380, Aurora kinase inhibitors, including, fore example, VX-680, and p38 MAP kinase inhibitors, including, for example, BIRB-796. The treatment regimen can include administration of a higher dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, a reduced dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, or an unchanged dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent.

Amounts of dasatinib (BMS-354825) effective to treat cancer would broadly range between about 10 mg. and about 150 mg. per day, more generally range between about 35 mg. and about 140 mg. per day, and preferably between about 70 mg. and about 140 mg. per day (administered orally twice a day). The rationale for the preferred dose range is based upon BMS-354825 dosing for CML and the clinical pharmacology data presented in “Dasatinib (BMS-354825) Oncologic Drug Advisory Committee (ODAC) briefing document, NDA-21-986, in which the Cmax was between approximately 60-120 nM. It is further envisioned that BMS-354825 may be administered either alone or in conjunction with therapies aimed at treating or preventing cancer.

The present invention also encompasses a pharmaceutical composition useful in the treatment of cancer, comprising the administration of a therapeutically effective amount of the compound of the present invention, either alone or in combination with other compounds useful in the treatment of cancer, with or without pharmaceutically acceptable carriers or diluents. The compositions of the present invention may further comprise one or more pharmaceutically acceptable additional ingredient(s) such as alum, stabilizers, antimicrobial agents, buffers, coloring agents, flavoring agents, adjuvants, and the like. The compositions of the present invention may be administered orally or parenterally including the intravenous, intramuscular, intraperitoneal, subcutaneous, rectal and topical routes of administration.

For oral use, the compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

The combinations of the present invention may also be used in conjunction with other well known therapies that are selected for their particular usefulness against the condition that is being treated.

The effective amount of the compounds of the combination of the present invention may be determined by one of ordinary skill in the art, and includes exemplary dosage amounts for an adult human of from about 0.1 to 2 mg/kg of body weight of active compound per day, preferably at a dose from 0.1 to 2 mg/kg of body weight which may be administered in a single dose or in the form of individual divided doses, such as from 1 to 2 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Subjects for treatment include animals, most preferably mammalian species such as humans, and domestic animals such as dogs, cats and the like, subject to protein tyrosine kinase-associated disorders.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

The therapeutic agent(s) can be administered according to therapeutic protocols well known in the art.

It will be apparent to those skilled in the art that the administration of the therapeutic agent(s) can be varied depending on the disease being treated and the known effects of the therapeutic agent(s). Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

The invention also relates to a kit, wherein the agents/compounds are disposed in separate containers. The invention also relates to a kit according to any of the foregoing, further comprising integrally thereto or as one or more separate documents, information pertaining to the contents or the kit and the use of the agents/inhibitors. The invention also relates to a kit according to any of the foregoing, wherein the compositions are formulated for reconstitution in a diluent. The invention also relates to a kit according to any of the foregoing, further comprising a container of sterile diluent. The invention also relates to a kit according to any of the foregoing, wherein said compositions are disposed in vials under partial vacuum sealed by a septum and suitable for reconstitution to form a formulation effective for parental administration.

Exemplary Indications, Conditions, Diseases, and Disorders:

The present invention provides methods of determining the responsiveness of an individual having a proliferative disorder of the lung to a certain treatment regimen and methods of treating an individual having a proliferative disorder of the lung.

The term “proliferative disorder of the lung” as used herein is inclusive of lung cancer, non-small cell lung cancer, etc. This term may also be construed to include additional lung disorders, including, but not limited to ARDS, emphysema, cystic fibrosis, interstitial lung disease, chronic obstructive pulmonary disease, bronchitis, lymphangioleiomyomatosis, pneumonitis, eosinophilic pneumonias, granulomatosis, pulmonary infarction, pulmonary fibrosis, pneumoconiosis, alveolar hemorrhage, neoplasms, lung abscesses, empyema, and increased susceptibility to lung infections (e.g., immunocompromised, HIV, etc.), pulmonary infections: pneumonia, bacterial pneumonia, viral pneumonia (for example, as caused by Influenza virus, Respiratory syncytial virus, Parainfluenza virus, Adenovirus, Coxsackievirus, Cytomegalovirus, Herpes simplex virus, Hantavirus, etc.), mycobacteria pneumonia (for example, as caused by Mycobacterium tuberculosis, etc.) mycoplasma pneumonia, fungal pneumonia (for example, as caused by Pneumocystis carinii, Histoplasma capsulatum, Coccidioides immitis, Blastomyces dermatitidis, Candida sp., Cryptococcus neoformans, Aspergillus sp., Zygomycetes, etc.), Legionnaires' Disease, Chlamydia pneumonia, aspiration pneumonia, Nocordia sp. infections, parasitic pneumonia (for example, as caused by Strongyloides, Toxoplasma gondii, etc.) necrotizing pneumonia, in addition to any other pulmonary disease and/or disorder (e.g., non-pneumonia).

Additional disorders included in the scope of the present invention include, for example, leukemias, including, for example, chronic myeloid leukemia, acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is T-ALL, chronic myeloid leukemia (CML), Ph+ ALL, AML, imatinib-resistant CML, imatinib-intolerant CML, accelerated CML, lymphoid blast phase CML.

A “solid tumor” includes, for example, sarcoma, melanoma, carcinoma, or other solid tumor cancer.

The terms “cancer”, “cancerous”, or “malignant” refer to or describe the physiological condition in mammals that is typically characterized by unregulated cell growth. Examples of cancer include, for example, lung cancer, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

A “mutant EGFR” encompasses a EGFR with an amino acid sequence that differs from wild type EGFR by one or more amino acid substitutions, additions or deletions. Such a mutant EGFR may preferably constitute an activating mutation, including, but not limited to mutations that selectively activate Akt and signal transducer and activator of transcription (STAT) pathways important in lung cancer cell survival.

“EGFR-dependent mutation” is used to describe an EGFR mutation in which cells have become dependent on the activated EGFR state for survival, and which may thus have increased sensitivity to the administration of a protein tyrosine kinase inhibitor relative to individuals harboring wild type EGFR. For example, a protein tyrosine kinase inhibitor compound can be used to treat a cancerous condition, which compound inhibits the activity of wild type EGFR which will inhibit proliferation and/or induce apoptosis of cancerous cells.

“EGFR status” refers to the status of the EGFR in the cell or cells under examination, or more particularly, the presence or absence of activating mutations or additions in EGFR of the cell and/or the identity of the particular mutation or addition in the EGFR. The status of EGFR changes from wildtype to a molecule containing activating mutations. Status is observed to change as a result of gene amplification and/or mutation. EGFR status can predict or result in changes in sensitivity to EGFR targeting agents, such as gefitinib and erlotinib. Additionally, cells may have mutations in EGFR that make them insensitive to agents acting on EGFR, but retain sensitivity to other agents, such as dasatinib, that do not target EGFR in a manner analogous to recognized EGFR targeting agents.

Treatment Regimens

The invention encompasses treatment methods based upon the demonstration that patients harboring different EGFR forms, i.e., wild type and activating EGFR, have varying degrees of resistance and/or sensitivity to N-(2-chloro-6-methylphenyl)-2-[[6-[4-(2-hydroxyethyl)-1-piperazinyl]-2-methyl-4-pyrimidinyl]amino]-5-thiazolecarboxamide, respectively. Thus the methods of the present invention can be used, for example, in determining whether or not to treat an individual with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; whether or not to treat an individual with a more aggressive dosage regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; or whether or not to treat an individual with combination therapy, i.e., a combination of tyrosine kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and additional protein tyrosine kinase inhibitors(s) (e.g., such as imatinib, AMN107, PD180970, GGP76030, AP23464, SKI 606, NS-187, and/or AZDO530); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a tubulin stabilizing agent (such as, for example, pacitaxol, epothilone, taxane, and the like.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and ATP non-competitive inhibitors ONO12380; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and Aurora kinase inhibitor VX-680; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and p38 MAP kinase inhibitor BIRB-796; any other combination disclosed herein.

The terms “treating”, “treatment” and “therapy” as used herein refer to curative therapy, prophylactic therapy, preventative therapy, and mitigating disease therapy.

In certain embodiments, the present invention provides a method of identifying whether a patient harbors the wild-type EGFR, or an activating EGFR mutation, in a mammalian cell, wherein the wild type EGFR polynucleotide is associated with at least partial resistance to inhibition of protein tyrosine kinase activity by N-(2-chloro-6-methylphenyl)-2-[[6-[4-(2-hydroxyethyl)-1-piperazinyl]-2-methyl-4-pyrimidinyl]amino]-5-thiazolecarboxamide, the method comprising determining the sequence of at least one EGFR polynucleotide expressed by the mammalian cell and comparing the sequence of the EGFR polynucleotide to the wild type EGFR polynucleotide sequence.

In the method disclosed above, the mammalian cell can be a human cancer cell. The human cancer cell can be one obtained from an individual treated having a proliferative disorder of the lung.

For use herein, a protein tyrosine kinase inhibitor refers to any molecule or compound that can partially inhibit ECR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl (see, e.g., Zimmerman et al., Bioorg, Med. Chem. Lett. 7, 187 (1997)). All of these inhibitors are encompassed within the term a BCR-ABL inhibitor. Imatinib, one of these inhibitors, also known as STI-571 (formerly referred to as Novartis test compound CGP 57148 and also known as Gleevec), has been successfully tested in clinical trail a therapeutic agent for CML. AMN107, is another BCR-ABL kinase inhibitor that was designed to fit into the ATP-binding site of the BCR-ABL protein with higher affinity than imatinib. In addition to being more potent than imatinib (IC50<30 nM) against wild-type BCR-ABL, AMN107 is also significantly active against 32/33 imatinib-resistant BCR-ABL mutants. In preclinical studies, AMN107 demonstrated activity in vitro and in vivo against wild-type and imatinib-resistant BCR-ABL-expressing cells. In phase I/II clinical trials, AMN107 has produced haematological and cytogenetic responses in CML patients, who either did not initially respond to imatinib or developed imatinib resistance (Weisberg et al., British Journal of Cancer (2006) 94, 1765-1769, incorporated herein by reference in its entirety and for all purposes). SKI-606, NS-187, AZD0530, PD180970, CGP76030, and AP23464 are all examples of kinase inhibitors that can be used in the present invention. SKI-606 is a 4-anilino-3-quinolinecarbonitrile inhibitor of Abl that has demonstrated potent antiproliferative activity against CML cell (Golas et al., Cancer Research (2003) 63, 375-381). AZDO530 is a dual Abl/Src kinase inhibitor that is in ongoing clinical trials for the treatment of solid tumors and leukemia (Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004). PD180970 is a pyrido[2,3-d]pyrimidine derivative that has been shown to inhibit BCR-ABL and induce apoptosis in BCR-ABL expressing leukemic cells (Rosee et al., Cancer Research (2002) 62, 7149-7153). CGP76030 is dual-specific Src and Abl kinase inhibitor shown to inhibit the growth and survival of cells expressing imatinib-resistant BCR-ABL kinases (Warmuth et al., Blood, (2003) 101(2), 664-672). AP23464 is an ATP-based kinase inhibitor that has been shown to inhibit imatinib-resistant BCR-ABL mutants (O'Hare et al., Clin Cancer Res (2005) 11(19), 6987-6993). NS-187 is a selective dual Bcr-Abl/Lyn tyrosine kinase inhibitor that has been shown to inhibit imatinib-resistant BCR-ABL mutants (Kimura et al., Blood, 106(12):3948-3954 (2005)).

A “farnysyl transferase inhibitor” can be any compound or molecule that inhibits farnysyl transferase. The farnysyl transferase inhibitor can have formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (II) is a cytotoxic FT inhibitor which is known to kill non-proliferating cancer cells preferentially. The compound of formula (II) can further be useful in killing stem cells.

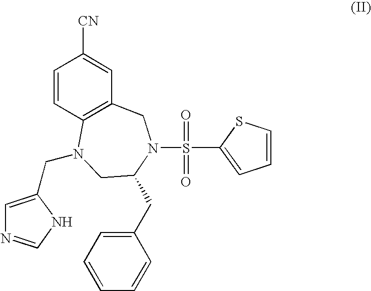

The compound of formula (U), its preparation, and uses thereof are described in U.S. Pat. No. 6,011,029, which is herein incorporated by reference in its entirety and for all purposes. Uses of the compound of formula (II) are also described in WO2004/015130, published Feb. 19, 2004, which is herein incorporated by reference in its entirety and for all purposes.

For use herein, combination therapy refers to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof with a second therapy at such time that both the second therapy and N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, will have a therapeutic effect. Such administration can involve concurrent (i.e., at the same time), prior, or subsequent administration of the second therapy with respect to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof.

Treatment regimens can also be established based upon the presence of one or more mutant BCR-ABL kinases disclosed herein. For example, the invention encompasses screening cells from an individual who may suffer from, or is suffering from, a disorder that is commonly treated with a kinase inhibitor. Such a disorder can include myeloid leukemia or disorders associated therewith, or cancers described herein. The cells of an individual are screened, using methods known in the art, for identification of a mutation in a BCR-ABL kinase. Mutations of interest are those that result in BCR-ABL kinase being constitutively activated. Specific mutations may include, for example, F317I (wherein the phenylalanine at position 317 is replaced with an isoleucine), and T315A (wherein the threonine at position 315 is replaced with an alanine). Other mutations include, for example, E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, 1314V, T3151, E316G, F317L, M318T, Y320C, Y320H, G321E, D325H, Y326C, L327P, R328K, E329V, Q333L, A337V, V339G, L342E, M343V, M343T, A344T, A344V, 1347V, A350T, M351T, E352A, E352K, E355G, K357E, N358D, N358S, F359V, F359C, F359I, I360K, I360T, L364H, L3641, E373K, N374D, K378R, V3791, A380T, A380V, D381G, F382L, L387M, M388L, T389S, T392A, T394A, A395G, H396K, H396R, A399G, P402T, T406A, S417Y, F486S or any combination thereof, i.e., M244V, G250E, Q252H, Q252R, Y253F, Y253H, E255K, E255V, T315I, F317L, M351T, E355G, F359V, H396R, F486S and any combination thereof; M244V, E279K, F359C, F359I, L364I, L387M, F486S and any combination thereof; and L248R, Q252H, E255K, V299L, T3151, F317V, F317L, F317S and any combination thereof.

If an activating BCR-ABL kinase mutation is found in the cells from said individual, treatment regimens can be developed appropriately. For example, an identified mutation can indicate that said cells are or will become at least partially resistant to commonly used kinase inhibitors. For example, a F317I or T315A mutation can indicate that the cells in an individual are or are expected to become at least partially resistant to treatment with a kinase inhibitor such as N-(2-chloro-6-methylphenyl)-2-[[6-[4-(2-hydroxyethyl)-1-piperazinyl]-2-methyl-4-pyrimidinyl]amino]-5-thiazolecarboxamide. As disclosed herein, in such cases, treatment can include the use of an increased dosing frequency or increased dosage of N-(2-chloro-6-methylphenyl)-2-[[6-[4-(2-hydroxyethyl)-1-piperazinyl]-2-methyl-4-pyrimidinyl]amino]-5-thiazolecarboxamide or a salt, hydrate, or solvate thereof, a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and another kinase inhibitor drug such as imatinib, AMN107, PD180970, GGP76030, AP23464, SKI 606, and/or AZD0530; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; any other combination disclosed herein; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein.

A method of determining the responsiveness of an individual suffering from a protein tyrosine kinase-associated disorder to a combination of kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide and imatinib, is disclosed herein. For example, an individual can be determined to be a positive responder (or cells from said individual would be expected to have a degree of sensitivity) to a certain kinase inhibitor based upon the presence of a mutant BCR-ABL kinase. Cells that exhibit certain mutations at amino acid positions 315 and 317 of BCR-ABL kinase, for example, can develop at least partial resistance to N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof. Therefore, individuals suffering from a protein tyrosine kinase-associated disorder whose cells exhibit such a mutation are or would be expected to be partially negative responders to a particular treatment regimen with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof but a positive responder to a more aggressive treatment regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof or to combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and imatinib or other therapy.

Irrespective of whether wild type EGFR, and/or a BCR-ABL mutation is present, or even any other mutant that may require increased administration of a protein tyrosine kinase inhibitor, an increased level of N-(2-chloro-6-methylphenyl)-2-[[6-[4-(2-hydroxyethyl)-1-piperazinyl]-2-methyl-4-pyrimidinyl]amino]-5-thiazolecarboxamide would be about 10, 20, 30, 40, 50, 60, 70, 80, 90, or 95% more than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual, or about 1.5×, 2×, 2.5×, 3×, 3.5×, 4×, 4.5×, 5×, 6×, 7×, 8×, 9×, or 10× more N-(2-chloro-6-methylphenyl)-2-[[6-[4-(2-hydroxyethyl)-1-piperazinyl]-2-methyl-4-pyrimidinyl]amino]-5-thiazolecarboxamide than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual.

Additionally, dosage regimens can be further adapted based upon the presence of additional amino acid mutation in a BCR-ABL kinase. As described herein, a mutation in E279K, F359C, F3591, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, 1293T, P296S, L298M, L298P, V299L, Q300R, G303E, V304A, V304D, C305S, C305Y, T306A, F311L, 1314V, T3151, E316G, F317L, M318T, Y320C, Y320H, G321E, D325H, Y326C, L327P, R328K, E329V, Q333L, A337V, V339G, L342E, M343V, M343T, A344T, A344V, 1347V, A350T, M351T, E352A, E352K, E355G, K357E, N358D, N358S, F359V, F359C, F359I, I360K, I360T, L364H, L364I, E373K, N374D, K378R, V3791, A380T, A380V, D381G, F382L, L387M, M388L, T389S, T392A, T394A, A395G, H396K, H396R, A399G, P402T, T406A, S417Y, F486S, or any combination thereof can indicate that the BCR-ABL kinase has developed at least partial resistance to therapy with a protein kinase inhibitor such as imitinab.

A therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof can be orally administered as an acid salt of N-(2-chloro-6-methylphenyl)-2-[[6-[4-(2-hydroxyethyl)-1-piperazinyl]-2-methyl-4-pyrimidinyl]amino]-5-thiazolecarboxamide. The actual dosage employed can be varied depending upon the requirements of the patient and the severity of the condition being treated. Determination of the proper dosage for a particular situation is within the skill of the art. The effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof (and Compound I salt) can be determined by one of ordinary skill in the art, and includes exemplary dosage amounts for an adult human of from about 0.05 to about 100 mg/kg of body weight of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, per day, which can be administered in a single dose or in the form of individual divided doses, such as from 1, 2, 3, or 4 times per day. In certain embodiments, N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof is administered 2 times per day at 70 mg. Alternatively, it can be dosed at, for example, 50, 70, 90, 100, 110, or 120 BID, or 100, 140, or 180 once daily. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to Compound II or any combination of Compound I and II, or any combination disclosed herein.

A treatment regimen is a course of therapy administered to an individual suffering from a protein kinase associated disorder that can include treatment with one or more kinase inhibitors, as well as other therapies such as radiation and/or other agents (i.e., combination therapy). When more than one therapy is administered, the therapies can be administered concurrently or consecutively (for example, more than one kinase inhibitor can be administered together or at different times, on a different schedule). Administration of more than one therapy can be at different times (i.e., consecutively) and still be part of the same treatment regimen. As disclosed herein, for example, cells from an individual suffering from a protein kinase associated disorder can be found to develop at least partial resistance to N-(2-chloro-6-methylphenyl)-2-[[6-[4-(2-hydroxyethyl)-1-piperazinyl]-2-methyl-4-pyrimidinyl]amino]-5-thiazolecarboxamide. Based upon the present discovery that such cells can be sensitive to combination therapy or a more aggressive dosage or dosing regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, a treatment regimen can be established that includes treatment with the combination either as a monotherapy, or in combination with another kinase inhibitor, or in combination with another agent as disclosed herein. Additionally, the combination can be administered with radiation or other known treatments.

Therefore the present invention includes a method for establishing a treatment regimen for an individual suffering from a proliferative disorder of the lung, and/or a protein tyrosine kinase associated disorder or treating an individual suffering from a protein tyrosine kinase disorder comprising determining whether a biological sample obtained from an individual has either a mutant or wild type EGFR, and administering to the subject an appropriate treatment regimen based on whether the mutation is present. The determination can be made by any method known in the art, for example, by screening said sample of cells for the presence of at least one activating mutation in a EGFR sequence or by obtaining information from a secondary source that the individual has the specified EGFR mutation.

In practicing the many aspects of the invention herein, biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

Pharmaceutical compositions for use in the present invention can include compositions comprising one or a combination of protein tyrosine kinase inhibitors in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

Dosage regimens involving N-(2-chloro-6-methylphenyl)-2-[[6-[4-(2-hydroxyethyl)-1-piperazinyl]-2-methyl-4-pyrimidinyl]amino]-5-thiazolecarboxamide useful in practicing the present invention are described in U.S. Ser. No. 10/395,503, filed Mar. 24, 2003; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; which are hereby incorporated herein by reference in their entirety and for all purposes.

A “therapeutically effective amount” of an inhibitor of a wild type or mutant EGFR can be a function of the mutation present. One skilled in the art will appreciate the difference in sensitivity of the mutant BCR-ABL kinase cells and determine a therapeutically effective dose accordingly.

Examples of predicted therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide that may be warranted based upon the relative sensitivity of wild type EGFR to N-(2-chloro-6-methylphenyl)-2-[[6-[4-(2-hydroxyethyl)-1-piperazinyl]-2-methyl-4-pyrimidinyl]amino]-5-thiazolecarboxamide compared to mutant EGFR can be determined using various in vitro biochemical assays including cellular proliferation, EGFRphosphorylation, peptide substrate phosphorylation, and/or autophosphorylation assays. For example, approximate therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide can be calculated based upon multiplying the typical dose with the fold change in sensitivity in anyone or more of these assays for each EGFR form tested. For example, if wild type EGFR required 14 fold higher level of protein tyrosine kinase inhibitor to achieve an efficacious level of cell death relative to an activating EGFR mutation, a therapeutically relevant dose of N-(2-chloro-6-methylphenyl)-2-[[6-[4-(2-hydroxyethyl)-1-piperazinyl]-2-methyl-4-pyrimidinyl]amino]-5-thiazolecarboxamide for patients harboring this mutation could range, for example, anywhere from 1 to 14 fold higher than the typical dose. Accordingly, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide for any form of EGFR can be, for example, 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 folder higher than the prescribed dose. Alternatively, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide can be, for example, 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose.

According to the present invention, dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. See, e.g., the latest Remington's (Remington's Pharmaceutical Science, Mack Publishing Company, Easton, Pa.)

Additional Terminolgy:

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Therapeutically effective amount” refers to an amount of a compound of the present invention alone or an amount of the combination of compounds claimed or an amount of a compound of the present invention in combination with other active ingredients effective to treat the diseases described herein.

As used in relation to the invention, the term “treating” or “treatment” and the like should be taken broadly. They should not be taken to imply that a subject is treated to total recovery. Accordingly, these terms include amelioration of the symptoms or severity of a particular condition or preventing or otherwise reducing the risk of further development of a particular condition.

It should be appreciated that methods of the invention may be applicable to various species of subjects, preferably mammals, more preferably humans.

As used herein, the compounds of the present invention include the pharmaceutically acceptable derivatives thereof.

A “pharmaceutically-acceptable derivative” denotes any salt, hydrate, solvate of ester of a compound of this invention, or any other compound which upon administration to a patient is capable of providing (directly or indirectly), such as a prodrug, a compound of this invention, or a metabolite or residue thereof.

The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, aistidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. When a basic group and an acid group are present in the same molecule, a compound of the invention may also form internal salts.

The term “prodrug,” as used herein, refers to compounds which are rapidly transformed in vivo to the parent compound of the above formula, for example, by hydrolysis in blood. A thorough discussion is provided by T. Higuchi and V. Stella, “Pro-drugs as Novel Delivery systems,” Vol. 14 of the A. C. S. Symposium Series, and in Edward B. Roche, ed., “Bioreversible Carriers in Drug Design,” American Pharmaceutical Association and Pergamon Press, 1987, both of which are incorporated herein by reference.

The invention is described below in examples which are intended to further describe the invention without limitation to its scope.

### Example 1

### Materials and Methods

Cell lines and cell culture. Human lung cancer cell lines were maintained in RPMI 1640 plus 5% bovine calf serum (BCS). H3255 cells were provided by Dr. Pasi Janne (Dana-Farber, Boston, Mass.) and grown in ACL-4 medium (12). HCC827 cells were provided by Dr. Jon Kurie (M. D. Anderson, Houston, Tex.). PC9 cells were provided by Dr. Matthew Lazzara (Massachusetts Institute of Technology, Boston, Mass.). Stock solutions of gefitinib and dasatinib in 100% DMSO were diluted directly into the medium to indicated concentrations. Gefitinib was provided by AstraZeneca (Wilmington, Del.) and dasatinib by Bristol-Myers Squibb Oncology (Princeton, N.J.). For cell transfection experiments, 2×10⁶ HEK293 cells in a 6-cm dish maintained in DMEM/10% BCS were transfected with 1 Ag plasmid DNA for 3 hours using LipofectAMINE 2000 (Invitrogen, Carlsbad, Calif.) and then allowed to go 24 hours before being treated with inhibitors.

Cytotoxicity and apoptosis assays. Cytotoxicity assays [3-(4,5-dimethylthiazol-2-yl)-2,5-diphenyltetrazolium bromide] were done according to the manufacturer's recommendations (Roche, Indianapolis, Ind.). Cells (5×10³) in medium with 5% BCS were placed into single wells in a 96-well plate and exposed to indicated agents, and viability was assessed after 72 hours. Data presented represent three separate experiments with eight data points per concentration per experiment. Apoptosis [PharMingen (San Diego, Calif.) Apo-BrdU kit] and cell cycle changes (propidium iodide staining and flow cytometry) were assayed as before (13). Data are expressed as mean±SD.

Invasion assays. Boyden chambers (8 μm pores; Costar, Fisher, Corning, N.Y.) were loaded with 10 Ag growth factor-depleted/reduced Matrigel (BD Biosciences, San Diego, Calif.) and air dried overnight. Cells (100,000) in medium plus 0.1% bovine serum albumin were loaded onto the top chamber, whereas complete medium was added to lower chamber, and chambers were loaded in duplicate and placed back into the incubator. After 22 hours, the filters were removed, wiped with a cotton swab to remove Matrigel and noninvading cells, and stained with DiffQuick. Five fields were counted for invading cells per filter.

Protein expression analysis. Cell lysates were prepared using radio-immunoprecipitation assay buffer [10 mmol/L Tris (pH 7.4), 100 mmol/L NaCl, 1 mmol/L EDTA, 1 mmol/L EGTA, 1 mmol/L NaF, 20 mmol/L Na₄P₂0₇, 2 mmol/L Na₃VO₄, 0.1% SDS, 0.5% sodium deoxycholate, 1% Triton X-100, 10% glycerol, 1 mmol/L phenylmethylsulfonyl fluoride, 60 μg/mL aprotinin, 10 μg/mL leupeptin, 1 μg/mL pepstatin], normalized for total protein content (50 μg), and subjected to SDS-PAGE. Primary antibodies used in these studies consisted of phosphotyrosine (pTyr)⁸⁴⁵ EGFR, pTyr¹⁰⁶⁸ EGFR(pEGFR), total EGFR, pSer⁴⁷³ Akt (pAkt), total Akt, pTyr⁷⁰⁵ STAT3 (pSTAT3), total STAT3, pTyr⁴¹⁶ Src (pSrc) family, c-Src, pTyr⁸⁶¹ FAK (pFAK), total FAK, p42/44 extracellular signal-regulated kinase (ERK), total ERK, and cleaved poly(ADP-ribose) polymerase (PARP; Cell Signaling, Danvers, Mass.). Cyclins D1 and D3 and p27 antibodies have been described previously (14).

### Example 2

### Dasatinib Sensitivity of NSCLC Cells Harboring EGFR Mutations

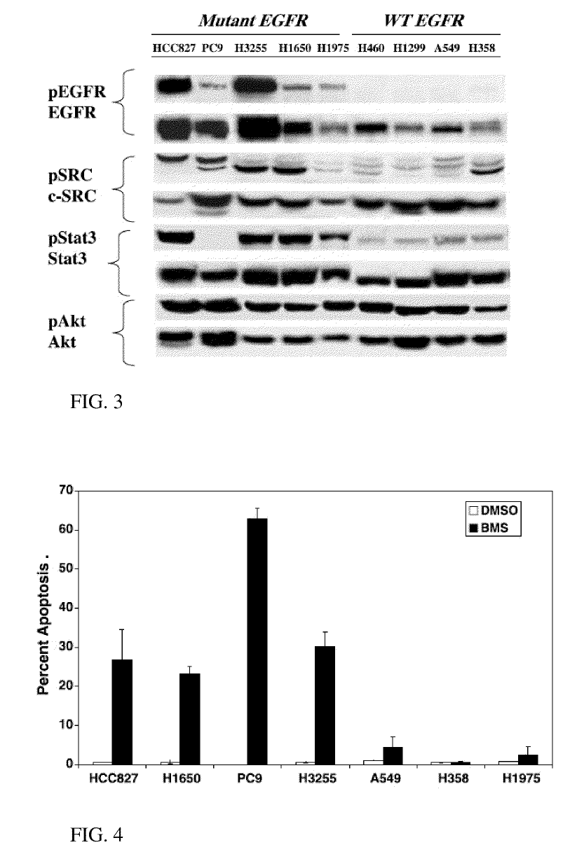

To assess dasatinib sensitivity of non-small cell lung cancer (NSCLC) cells harboring distinct EGFR mutations, cell lines containing the L858R mutation (H3255), L858R+T790M (H1975), and deletion mutation (HCC827, PC9, and H1650) along with cell lines with WT EGFR(H460, H358, H1299, and A549) were exposed to increasing concentrations of dasatinib and cell viability was assessed. As shown in FIG. 1, mutant EGFR cells are sensitive to dasatinib with an approximate IC₅₀ of 100 to 250 nmol/L, whereas WT EGFR and H1975 cells are resistant to dasatinib. (IC₅₀, >10 μmol/L). Dasatinib completely inhibits autophosphorylation of Tyr⁴¹⁶ on Src family members at a concentration of 50 nmol/L in H1650 cells (lower concentrations are not evaluated). An untreated group of parallel cells was evaluated for activated EGFR, Src family kinases, STAT3, and Akt. An antibody reflecting autophosphorylation of pTyr⁴¹⁶ on Src family proteins recognizes several distinct bands in the 56- to 60-kDa region with the suggestion of more expression in cells sensitive to dasatinib. Cell lines with mutant EGFR(H3255, H1650, PC9, HCC827, and H1975) were found to have enhanced levels of pEGFR and pSTAT3 compared with WT EGFR cells (H460, H358, H1299, and A549), with PC9 being the exception because it has undetectable pSTAT3 expression (FIG. 3).

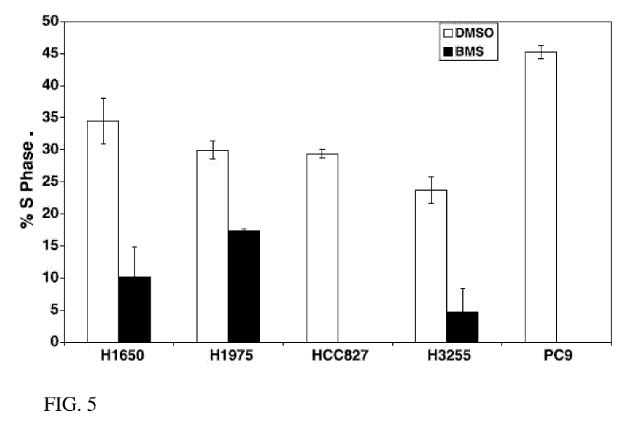

To assess how dasatinib affects cell viability, EGFR-mutant cell lines were assayed for changes in cell cycle and apoptosis. Dasatinib resulted in apoptosis in cells with EGFR mutants sensitive to gefitinib (H3255, H1650, HCC827, and PC9), whereas minimal apoptosis was observed in WT EGFR cells (A549 and H358) or in gefitinib resistant H1975 cells (FIG. 4). In addition to undergoing apoptosis, dasatinib inhibits DNA synthesis in cells with EGFR mutation, including H1975 (FIG. 5).

### Example 3

### Effect of Dasatinib and Gefitinib on Cell Viability in Cells with Gefitinib-Sensitive or Gefitinib-Resistant EGFR Mutation

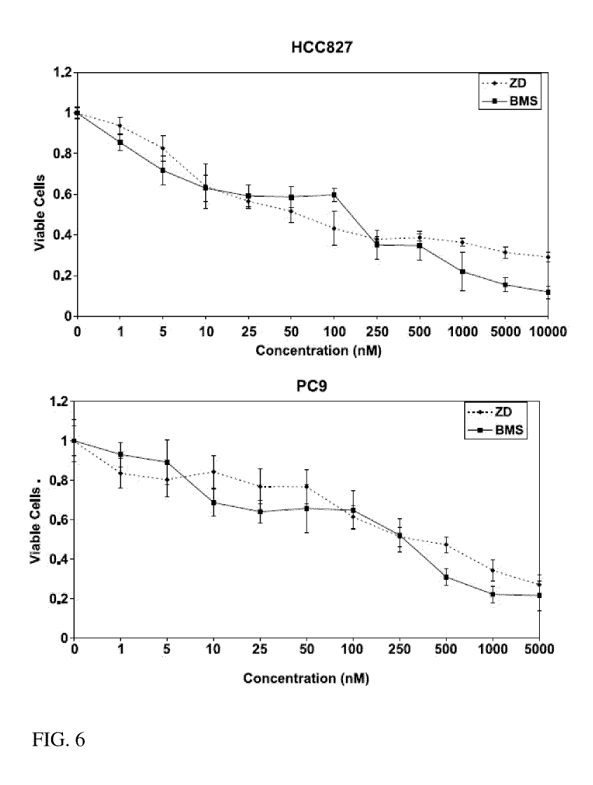

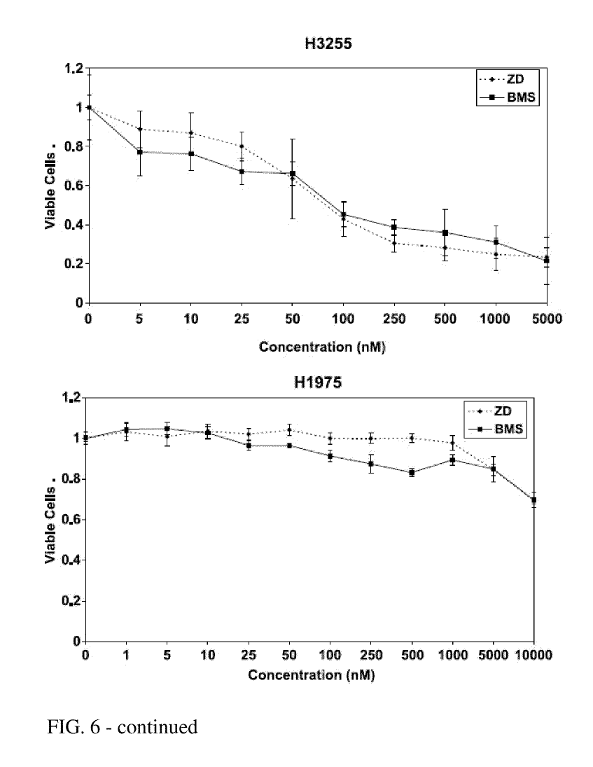

The effect of dasatinib and gefitinib on cell viability in cells with gefitinib-sensitive or gefitinib-resistant EGFR mutation was compared. Because cell growth conditions can affect sensitivity of cells to gefitinib, cell viability assays were repeated comparing dasatinib with gefitinib. In these cells, changes in cell viability as a function of concentration of inhibitor were similar to both gefitinib and dasatinib, with H1650 being the one exception because dasatinib inhibited cell viability more than gefitinib when grown in 5% BCS (FIG. 6).

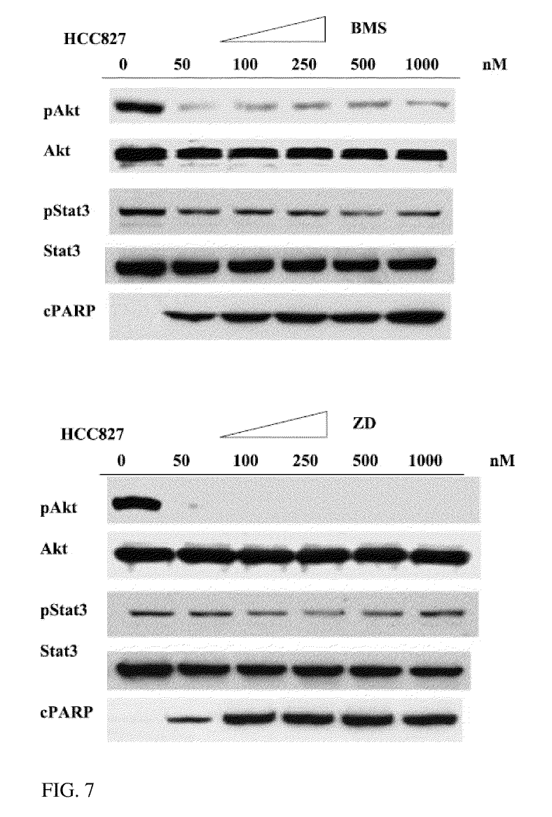

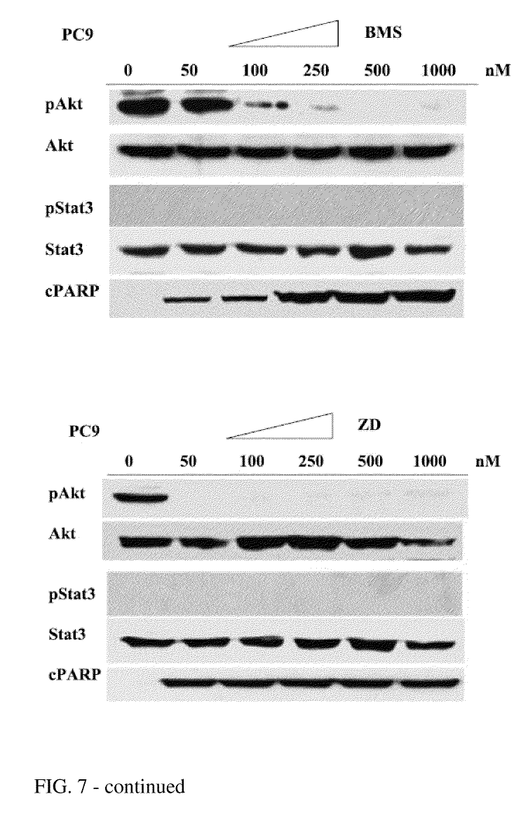

The effect of dasatinib on EGFR-mediated survival signaling through STAT3 and Akt was examined. The choice of these molecules was based on their role in mutant EGFR-dependent survival signaling as well as being downstream targets for Src signaling (Sordella R, et al., Science 2004; 305:1163-7; Amann J, et al., Cancer Res 2005; 65:226-35; Tracy S, et al., Cancer Res 2004; 64:7241-4; Martin G S. Nat Rev Mol Cell Biol 2001; 2:467-75). Cells were exposed to increasing concentrations of gefitinib or dasatinib for 24 hours and total proteins were evaluated for phosphorylated Akt and STAT3 as well as cleaved PARP indicative of apoptosis (FIG. 7). In HCC827 cells, dasatinib inhibits pAkt and induces PARP cleavage at 50 nmol/L, whereas modest changes are observed in pSTAT3. These results are similar in gefitinib-treated HCC827 cells. In PC9 cells, dasatinib exerts a dose-dependent inhibition of pAkt with associated changes in PARP cleavage, whereas a 50 nmol/L dose of gefitinib completely inhibits pAkt and induces PARP cleavage. In H3255 cells, dasatinib results in a concentration-dependent decrease of both pAkt and pSTAT3 with corresponding increase in PARP cleavage, whereas with associated induction of PARP cleavage. In H1650 cells grown in 5% BCS, dasatinib inhibits pAkt at 50 nmol/L with corresponding induction of PARP cleavage. Gefitinib has minimal effect on pAkt, and the degree of PARP cleavage is less corresponding to the higher IC₅₀ of gefitinib under these growth conditions. When the same cells are grown in low serum with exogenous EGF, both dasatinib and gefitinib inhibit pAkt at 50 nmol/L, but again the degree of PARP cleavage is higher in dasatinib-treated cells. No effect of either dasatinib or gefitinib is seen on pSTAT3 in any growth conditions. Finally, neither gefitinib nor dasatinib affects pAkt or pSTAT3 in H1975 cells, and no PARP cleavage is observed. These studies show that the induction of apoptosis by dasatinib is associated with reduction in activated Akt or STAT3 in a manner similar to that of gefitinib, although, in some cells, higher concentrations are necessary to see the effect on signaling and apoptosis.

### Example 4

### Effect of Gefitinib and Dasatinib on Src Phosphorylation Status

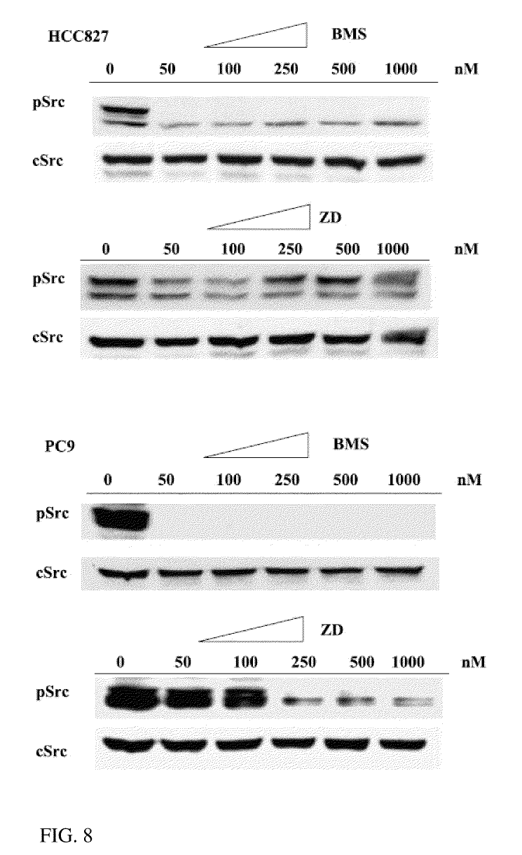

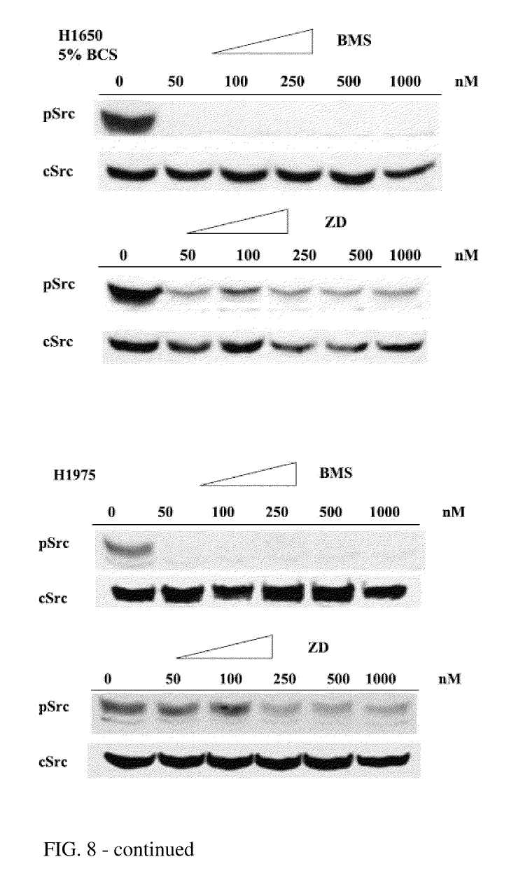

The effect of gefitinib and dasatinib on Src phosphorylation status was evaluated (FIG. 8). In HCC827 cells, dasatinib inhibits the lowest mobility pSrc band at 50 nmol/L, whereas no changes are seen in these cells with gefitinib. In PC9 cells, dasatinib inhibits pSrc at 50 nmol/L, whereas a decrease in pSrc is observed with 250 nmol/L gefitinib, but the effect is incomplete even at a 1 μmol/L concentration. Dasatinib completely inhibits pSrc at 50 nmol/L in H1650 cells, whereas the effect with gefitinib is incomplete. In H1975 cells, dasatinib completely inhibits the low levels of pSrc at 50 nmol/L, whereas minimal changes are observed with gefitinib. These results indicate that, in these EGFR-mutant cells, pSrc is largely maintained through EGFR-independent mechanisms that can be overcome by dasatinib.

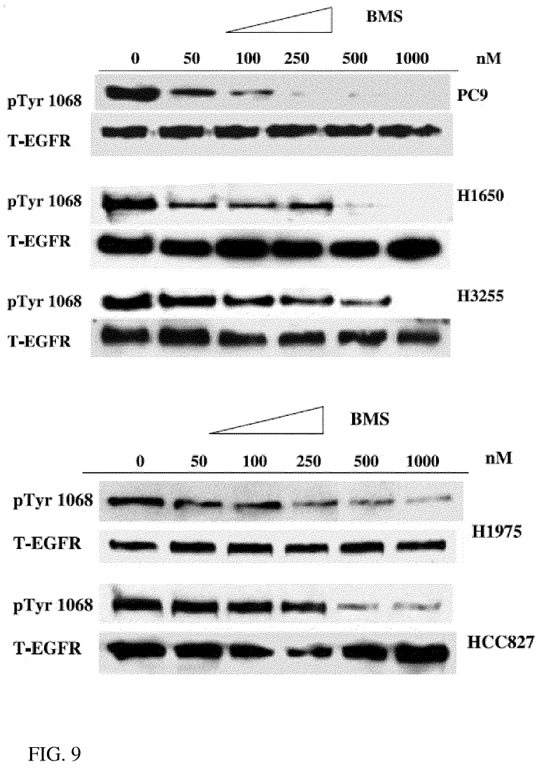

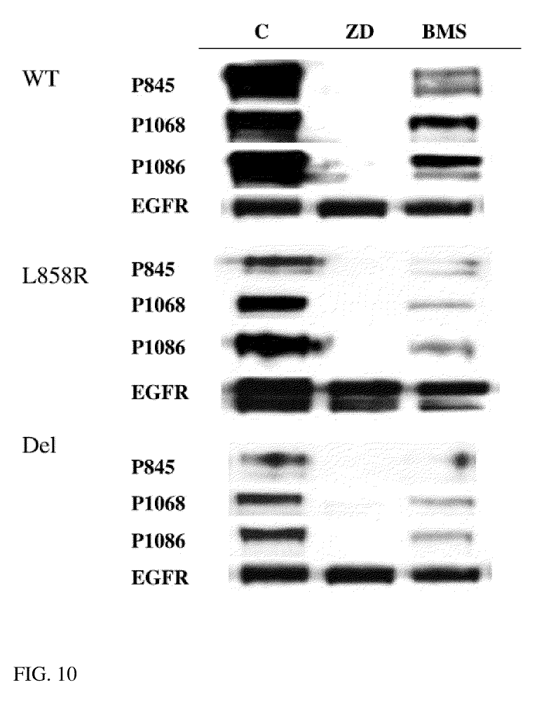

Because Src signaling has been shown to modify EGFR function and dasatinib has been suggested to bind EGFR, the effect of dasatinib on EGFR protein phosphorylation status was evaluated (Ishizawar R, and Parsons S J., Cancer Cell 2004; 6:209-14; Bromann P A, et al., Oncogene 2004; 23:7957-68; Carter T A, Wodicka L M, Shah N P, et al. Inhibition of drug-resistant mutants of ABL, KIT, and EGF receptor kinases. Proc Natl Acad Sci USA 2005; 102:11011-6). As shown in FIG. 9, dasatinib induces a concentration-dependent inhibition of EGFR phosphorylation status in the cell lines evaluated. To confirm these results, HEK293 cells that have low endogenous Erb expression were transfected with expression plasmids encoding WT EGFR, L858R EGFR, and del L747-E749; A750P EGFR, the cells exposed to either gefitinib or dasatinib, and then activated EGFR was evaluated using antibodies that specifically recognize distinct phosphotyrosines on EGFR (FIG. 10). As 1 μmol/L concentration of gefitinib completely inhibited EGFR phosphorylation. A 500 nmol/L dose of dasatinib inhibited EGFR phosphorylation, although not to the extent seen with gefitinib. These findings indicate that dasatinib may affect EGFR function through a combination of direct binding and inhibition and/or indirectly through Src inhibition (Ishizawar R, and Parsons S J., Cancer Cell 2004; 6:209-14; Bromann P A, et al., Oncogene 2004; 23:7957-68; Lombardo L J, et al., J Med Chem 2004; 47:6658-61).

### Example 5

### Effect of Src Inhibition on Lung Cancer Cells that do not have EGFR Mutations and do not Require EGFR for Survival

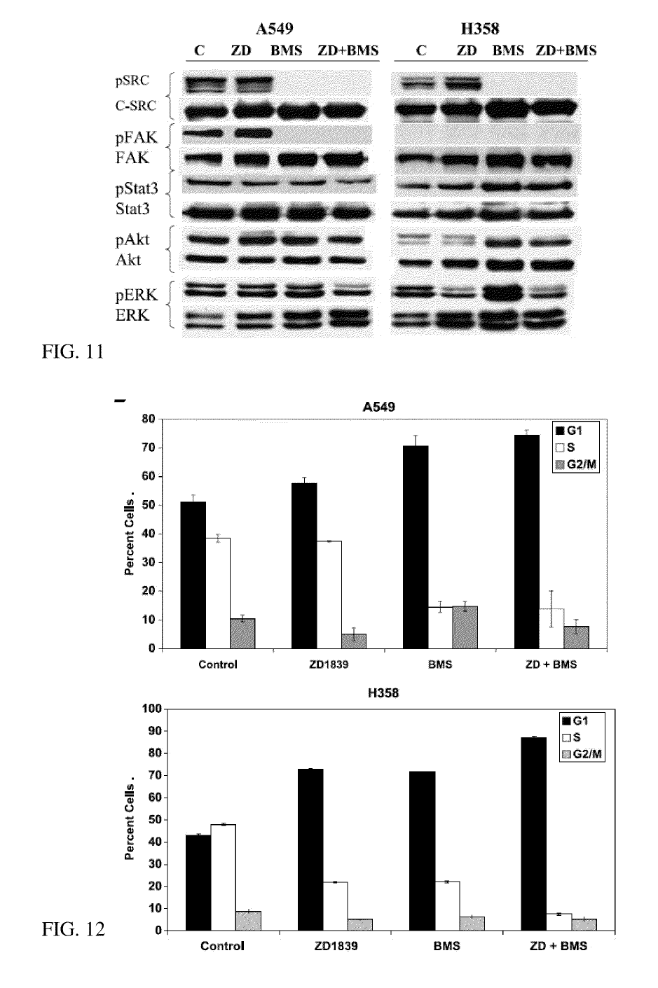

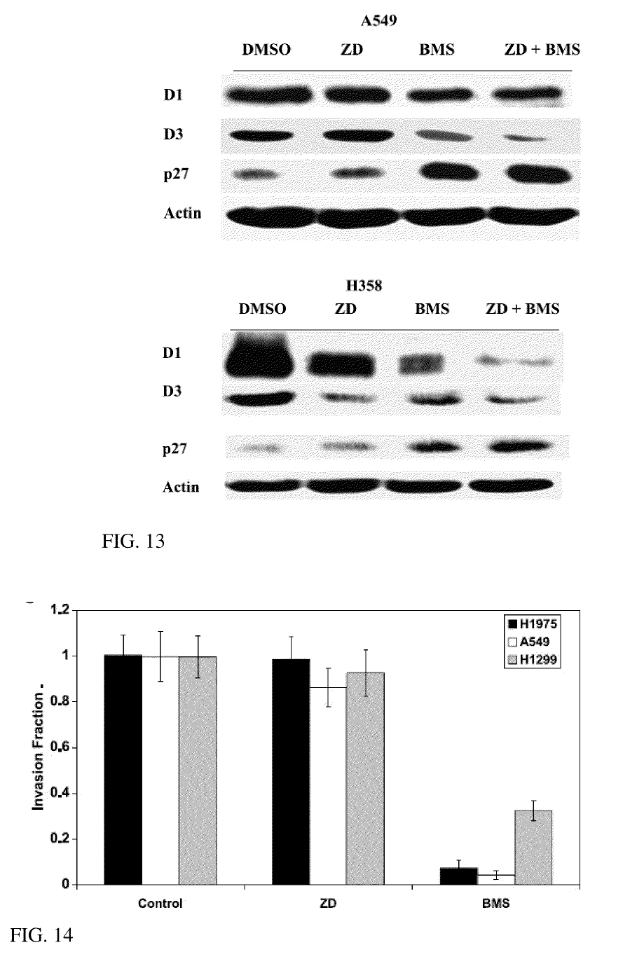

The effect of Src inhibition on lung cancer cells that do not have EGFR mutations and do not require EGFR for survival was evaluated (FIGS. 11-14). Dasatinib completely inhibits pSrc; however, a 1 μmol/L dose of gefitinib was unable to inhibit pSrc in A549 or H358 cells with WT EGFR. In A549 cells, we observed a reduction in pFAK but no significant reductions in pSTAT3, pAkt, or pERK. Similar results were observed in H358 cells, although these cells have no observable PFAK. As shown in FIG. 12, dasatinib results in cell cycle arrest characterized by increased G1 fraction and reduced S-phase fraction in A549 cells despite no effect with gefitinib.

The effect of dasatinib on key regulatory molecules cyclins D1 and D3 and p27 involved in G1-S cell cycle progression that can be regulated by Src was evaluated (Yeatman T J., Nat Rev Cancer 2004; 4:470-80; Martin G S. Nat Rev Mol Cell Biol 2001; 2:467-75). The G1 block resulting from dasatinib is associated with a decline in cyclin D3 and an increase in p27, whereas no changes in these critical cell cycle proteins are observed in gefitinib-treated cells nor are further changes in cyclin D3 or p27 observed in cells treated with both agents. On the other hand, H358 cells undergo G1 cell cycle arrest to the same extent between gefitinib and dasatinib, both compounds result in reduced cyclins D1 and D3 and increased p27 protein levels, and the combination results in further G1 cell cycle arrest and more pronounced changes in cyclins D1 and D3 and p27. Finally, consistent with the known role for Src in regulating tumor cell invasion, dasatinib has a significant inhibitory effect on tumor cell invasion in cells with WT EGFR(A549 and H1299) and gefitinib resistant EGFR(H1975) mutation, whereas gefitinib has no effect compared with control (FIG. 14).

### Example 6

### Effect of the SRC Tyrosine Kinase Inhibitor Dasatinib in Combination with Erlotinib and in Cells with Acquired Resistance to Erlotinib

SRC tyrosine kinase proteins can regulate oncogenic processes such as cell growth, survival, invasion, and angiogenesis. It is shown above that lung cancer cells dependent on EGFR for survival demonstrate increased sensitivity to dasatinib, a SRC tyrosine kinase inhibitor (TKI). The efficacy of dasatinib in combination with the EGFR TKI erlotinib in lung cancer cells with defined EGFR status was examined. Also examined was the effect of dasatinib on lung cancer cells with acquired resistance to erlotinib.

Lung cancer cells with defined EGFR status and sensitivity to erlotinib were evaluated for the combination effect of erlotinib and dasatinib using cell viability assays. Combination effects were evaluated by median dose effect method. Cells with EGFR mutation with acquired resistance to erlotinib were used to evaluate the effect of dasatinib on cell viability, cell cycle, and apoptosis. pSRC expression was examined in these cells by western analysis.

Using concentrations of gefitinib and dasatinib that result in concentration-dependent increases in apoptosis, the experiments suggest that dual EGFR/SRC inhibition additively or synergistically enhances apoptosis in PC9 lung cancer cells with EGFR mutation. The effect of dual EGFR/SRC TKI on lung cancer cells that do not have EGFR mutation, but nonetheless show some degree of sensitivity to EGFR TKI, was also examined. Synergy with erlotinib and dasatinib was identified in both H292 and H358 cells at lower concentrations of both TKI, while no effect was seen with either TKI in H441 cells in the dose range used. Both H292 and H358 cells show pSRC protein expression, while H441 cells have low levels of detectable pSRC.

Finally, lung cancer cells with EGFR mutation that are resistant to EGFR TKI were examined for the effect of dasatinib. These cells do not demonstrate significant amounts of apoptosis with dasatinib but they do undergo a dose-dependent G1 cell cycle arrest despite no observable effect on cell cycle with erlotinib.

The combination of erlotinib and dasatinib results in synergistic inhibition of viability and/or proliferation in lung cancer cells with dependence on EGFR for survival and/or growth. Resistance to erlotinib generally confers resistance to dasatinib, although higher concentrations of dasatinib can induce cell cycle arrest, some degree of apoptosis, and reduced cell viability.

### Example 7

### Sensitivity to EGFR Tyrosine Kinase Inhibitors Correlates with Sensitivity to Dasatinib

Lung cancer cell lines were exposed to either erlotinib (EGFR) tyrosine kinase inhibitor or dasatinib (SRC) Tyrosine kinase inhibitor. MTT assays were performed after 120 hours and IC₅₀ 's were calculated using the MTT assays. Table 1 presents the results of the assays. The results in Table 1 show that cells sensitive to an EGFR inhibitor are also sensitive to dasatinib. The results are consistent with our previously presented data indicating that EGFR status and sensitivity to EGFR tyrosine kinase inhibitors predicts sensitivity to dasatinib. Therefore, EGFR mutation analysis and EGFR gene amplification provide markers of sensitivity to EGFR inhibition and thus similarly predict sensitivity to dasatinib

TABLE 1

| | Histology | EGFR | TKI | SRC | Dasatinib |
|---|---|---|---|---|---|
| Cell | Status | Sensitivity | IC₅₀ (Nm) | Sensitivity | IC₅₀ (Nm) |
| H292 | Squamous WT | S | 63.4 | S | 28.4 |
| H358 | BAC WT | S | 199.1 | S | 27.4 |
| H441 | Adeno WT | R | >2,000 | R | >2,000 |
| A549 | Adeno WT | R | >2,000 | R | 413.3 |
| H460 | Large Cell WT | R | >2,000 | R | >2,000 |
| H1299 | Large Cell WT | R | >2,000 | S | 31.6 |
| H1648 | Adeno WT | S | 75.8 | S | 20.9 |
| H2122 | Adeno WT | R | 747.8 | R | 485.5 |
| H226 | Squamous WT | R | >2,000 | R | 708.7 |
| H157 | Squamous WT | R | >2,000 | S | 48.9 |
| H322 | BAC WT | S | 135.1 | S | 11.6 |
| H23 | Adeno WT | R | >2,000 | R | >2,000 |

EGFR TKI Sensitivity Definition: Reported IC₅₀ < or = 1 μM
SRC TKI Sensitivity Definition: IC₅₀ less than 100 nM

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of treating lung cancer in a subject comprising the steps of:
screening cancer cells of the subject to determine the EGFR status of the cells;
correlating the EGFR status of the subject's cells to the dasatinib treatment sensitivity associated with the EGFR status; and
administering a therapeutically-effective amount of dasatinib, or a pharmaceutically-acceptable derivative thereof, to the subject responsive to the correlated EGFR status of the subject's cells.

2. The method according to claim 1 wherein the sensitivity of cells to treatment with dasatinib associated with a defined EGFR status is determined prior to the screening step using a control cell population, whereby predetermining the sensitivity associated with a particular EGFR status enables rapid correlation of sensitivity of the cancer cell population following the screening step.

3. The method according to claim 1 further comprising the step of adjusting the dosage of dasatinib to be administered to the cancer cell population responsive to the correlation of the EGFR status of the subject's cells to the dasatinib treatment sensitivity associated with the EGFR status.

4. The method according to claim 1 wherein the lung cancer is non-small cell lung cancer.

5. The method according to claim 1 further comprising the step of administering one or more drugs selected from the group consisting of gefitinib, erlotinib and combinations thereof.

6. The method according to claim 1 further comprising the steps of:
correlating a biomarker of the screened cancer cells with the sensitivity of cells possessing that biomarker to treatment with a drug selected from the group of erlotinib, gefetinib and combinations thereof; and
administering the drug or combination thereof in combination with dasatinib.

7. The method according to claim 6 wherein both biomarkers are biomarkers of EGFR status.

8. A method of treating a proliferative disorder in a subject comprising the steps of:
screening cells of the subject to determine the EGFR status of the cells;
correlating the EGFR status of the subject's cells to the dasatinib treatment sensitivity associated with the EGFR status; and
administering a therapeutically-effective amount of dasatinib, or a pharmaceutically-acceptable derivative thereof, to the subject responsive to the correlated EGFR status of the subject.

9. The method according to claim 8 wherein the proliferative disorder is a disease selected from the group consisting of leukemias, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis, urticaria pigmentosa, cutaneous mastocytosis, solitary mastocytoma in human, dog mastocytoma, bullous mastocytosis, erythrodermic mastocytosis, teleangiectatic mastocytosis, mastocytosis with an associated hematological disorder, acute leukemia, myeloproliferative disorder associated with mastocytosis, mast cell leukemia, protein tyrosine kinase-associated disorders, squamous cell carcinoma, gastrointestinal stromal tumors, hematopoietic tumors of lymphoid lineage, hematopoietic tumors of myeloid lineage, tumors of mesenchymal origin, melanoma, seminoma, tetratocarcinoma, neuroblastoma, glioma, tumors of the central and peripheral nervous system, tumors of mesenchymal origin, xenoderma pigmentosum, keratoactanthoma, seminoma, thyroid follicular cancer, teratocarcinoma, chemotherapy refractory non-seminomatous germ-cell tumors, and Kaposi's sarcoma.

10. The method according to claim 8 wherein the proliferative disorder is a disease selected from the group consisting of leukemia, breast cancer, prostate cancer, lung cancer, colon cancer, melanoma, or solid tumors.

11. The method according to claim 10 wherein the leukemia is a leukemia selected from the group consisting of T-cell acute lymphoblastic leukemia (T-ALL), chronic myeloid leukemia (CML), Ph+ ALL, AML, imatinib-resistant CML, imatinib-intolerant CML, accelerated CML, and lymphoid blast phase CML.

12. The method according to claim 8 further comprising the step of adjusting the dosage of dasatinib to be administered to the cell population responsive to the correlation of the EGFR status of the subject's cells to the dasatinib treatment sensitivity associated with the EGFR status.

13. The method according to claim 8 further comprising the step of administering one or more drugs selected from the group consisting of gefitinib, erlotinib and combinations thereof.

14. A method of treating cancer in a subject comprising the steps of:
screening cancer cells of the subject to determine sensitivity to one or more EGFR tyrosine kinase inhibitors, whereby sensitivity to one or more EGFR tyrosine kinase inhibitors correlates with sensitivity to one or more SRC inhibitors; and
administering a therapeutically-effective amount of an SRC inhibitor to the subject responsive to the sensitivity to one or more EGFR tyrosine kinase inhibitors of the subject's cells.

15. The method according to claim 14 wherein the one of the one or more EGFR tyrosine kinase inhibitors is erlotinib.

16. The method according to claim 14 wherein the SRC inhibitor is dasatinib.

17. The method according to claim 14 wherein the cancer is leukemia, breast cancer, prostate cancer, lung cancer, colon cancer, melanoma, or solid tumors.

18. A method of treating cancer in a subject comprising the steps of:
screening cancer cells of the subject to determine the EGFR status of the cells;
correlating the EGFR status of the subject's cells to the dasatinib treatment sensitivity associated with the EGFR status; and
administering a therapeutically-effective amount of dasatinib, or a pharmaceutically-acceptable derivative thereof, to the subject responsive to the correlated EGFR status of the subject.

19. The method according to claim 18 further comprising the step of adjusting the dosage of dasatinib to be administered to the cancer cell population responsive to the correlation of the EGFR status of the subject's cells to the dasatinib treatment sensitivity associated with the EGFR status.

20. The method according to claim 18 further comprising the step of administering one or more EGFR inhibitors to the subject in combination with dasatinib.

21. The method according to claim 18 further comprising the steps of:
correlating a biomarker of the screened cancer cells with the sensitivity of cells possessing that biomarker to treatment one or more EGFR inhibitors; and
administering the one or more EGFR inhibitors in combination with dasatinib.

22. The method according to claim 21 wherein the one or more drugs selected from the group consisting of gefitinib, erlotinib and combinations thereof.

23. The method according to claim 21 wherein both biomarkers are biomarkers of EGFR status.

24. A method of treating a proliferative disorder in a subject comprising the steps of:
screening cells of the subject to determine the EGFR status of the cells;
correlating the EGFR status of the subject's cells to the SRC tyrosine inhibitor treatment sensitivity associated with the EGFR status; and
administering a therapeutically-effective amount of the SRC tyrosine inhibitor to the subject responsive to the correlated EGFR status of the subject.

25. The method according to claim 24 further comprising the step of administering one or more EGFR inhibitors to the subject in combination with the SRC tyrosine kinase inhibitor.
